(19) United States
(12) Patent Application Publication
(10) Pub. No.: US 2009/0156859 A1
(43) Pub. Date: Jun. 18, 2009

(54) Control of impurities in product glacial acetic acid of rhodium-catalyzed methanol carbonylation

(76) Inventors: Mark O. Scates, Houston, TX (US); G. Paull Torrence, Houston, TX (US)

Correspondence Address: Martin Connaughton;Celanese International Corporation, Clear Lake Technical Center, 9502 Bayport Blvd., Pasadena, TX, 77507, US

(21) Appl. No.: 11/804,922

(22) Filed: May 21, 2007

(51) Int. Cl. C07C 51/10

(52) U.S. Cl. 562/517

(57) ABSTRACT

The present invention relates to carbonylation of methanol, methyl acetate, dimethyl ether or mixtures thereof to produce glacial acetic acid, and more specifically to the manufacture of glacial acetic acid by the reaction of methanol, methyl acetate, dimethyl ether or mixtures thereof with carbon monoxide wherein the product glacial acetic acid contains low impurities.

### I. FIELD OF INVENTION

The present invention relates to carbonylation of methanol, methyl acetate, dimethyl ether or mixtures thereof to produce glacial acetic acid, and more specifically to the manufacture of glacial acetic acid by the reaction of methanol, methyl acetate, dimethyl ether or mixtures thereof with carbon monoxide wherein the product glacial acetic acid contains low impurities.

### II. BACKGROUND OF THE INVENTION

### A. Methanol Carbonylation to Produce Acetic Acid

For the production of acetic acid, there are three major commercialized processes, carbonylation process, acetaldehyde oxidation process, and liquid phase oxidation process, wherein the carbonylation process accounts for about 70% of the world manufacturing capacity. Among currently employed processes for synthesizing acetic acid, one of the most useful commercially is the catalyzed carbonylation of methanol with carbon monoxide as taught in U.S. Pat. No. 3,769,329 issued to Paulik et al. on Oct. 30, 1973. The carbonylation catalyst comprises rhodium, either dissolved or otherwise dispersed in a liquid reaction medium or else supported on an inert solid, along with a halogen-containing catalyst promoter as exemplified by methyl iodide. Generally, the reaction is conducted with the catalyst being dissolved in a liquid reaction medium, through which carbon monoxide gas is continuously bubbled. Paulik et al. disclose that water may be added to the reaction mixture to exert a beneficial effect upon the reaction rate, and water concentrations between about 14-15 wt % are typically used. This is the so-called “high water” carbonylation process.

An important aspect of the teachings of Paulik et al. is that water should also be present in the reaction mixture in order to attain a satisfactorily high reaction rate. The patentees exemplify a large number of reaction systems including a large number of applicable liquid reaction media. The general thrust of their teachings is, however, that a substantial quantity of water helps in attaining an adequately high reaction rate. The patentees teach furthermore that reducing the water content leads to the production of ester product as opposed to carboxylic acid. Considering specifically the carbonylation of methanol to acetic acid in a solvent comprising predominantly acetic acid and using the promoted catalyst taught by Paulik et al., it is taught in European Patent Application No. 0 055 618 that typically about 14-15 wt % water is present in the reaction medium of a typical acetic acid plant using this technology. It will be seen that in recovering acetic acid in anhydrous or nearly anhydrous form from such a reaction solvent, separating the acetic acid from this appreciable quantity of water involves a substantial expenditure of energy in distillation and/or additional processing steps such as solvent extraction, as well as enlarging some of the process equipment compared with that used in handling drier materials. Also Hjortkjaer and Jensen [Ind. Eng. Chem., Prod. Res. Dev. 16, 281-285 (1977)] have shown that increasing the water from 0 to 14 wt % water increases the reaction rate of methanol carbonylation. At above 14 wt % water, the reaction rate is unchanged.

In addition, as will be further explained hereinbelow, the catalyst tends to precipitate out of the reaction medium as employed in the process of Paulik et al., especially during the course of distillation operations to separate the product from the catalyst solution when the carbon monoxide content of the catalyst system is reduced (EP0055618). It is known that this tendency increases as the water content of the reaction medium is decreased. Thus, although it might appear obvious to try to operate the process of Paulik et al. at minimal water concentration in order to reduce the cost of handling a reaction product containing a substantial amount of water while still retaining enough water for an adequate reaction rate, the requirement for appreciable water in order to maintain catalyst activity and stability works against this end.

Other reaction systems are known in the art in which an alcohol such as methanol or an ether such as dimethyl ether or an ester such as methyl acetate can be carbonylated to an acid or ester derivative using special solvents such as aryl esters of the acid under substantially anhydrous reaction conditions. The product acid itself can be a component of the solvent system. Such a process is disclosed in U.S. Pat. No. 4,212,989 issued to Isshiki et al. on Jul. 15, 1975, with the catalytic metal being a member of the group consisting of rhodium, palladium, iridium, platinum, ruthenium, osmium, cobalt, iron, and nickel. A somewhat related patent is U.S. Pat. No. 4,336,399 issued to the same patentees, wherein a nickel-based catalyst system is employed. Considering U.S. Pat. No. 4,212,989 in particular, the relevance to the present invention is that the catalyst comprises both the catalytic metal, as exemplified by rhodium, along with what the patentees characterize as a promoter, such as the organic iodides employed by Paulik et al. as well as what the patentees characterize as an organic accelerating agent. The accelerating agents include a wide range of organic compounds of trivalent nitrogen, phosphorus, arsenic, and antimony. Sufficient accelerator is used to form a stoichiometric coordination compound with the catalytic metal. Where the solvent consists solely of acetic acid, or acetic acid mixed with the feedstock methanol, only the catalyst promoter is employed (without the accelerating agent), and complete yield data are not set forth. It is stated, however, that in this instance “large quantities” of water and hydrogen iodide were found in the product, which was contrary to the intent of the patentees.

European Published Patent Application No. 0 055 618 belonging to Monsanto Company discloses carbonylation of an alcohol using a catalyst comprising rhodium and an iodine or bromine component wherein precipitation of the catalyst during carbon monoxide-deficient conditions is alleviated by adding any of several named stabilizers. A substantial quantity of water, of the order of 14-15 wt %, was employed in the reaction medium. The stabilizers tested included simple iodide salts, but the more effective stabilizers appeared to be any of several types of specially-selected organic compounds. There is no teaching that the concentrations of methyl acetate and iodide salts are significant parameters in affecting the rate of carbonylation of methanol to produce acetic acid especially at low water concentrations. When an iodide salt is used as the stabilizer, the amount used is relatively small and the indication is that the primary criterion in selecting the concentration of iodide salt to be employed is the ratio of iodide to rhodium. That is, the patentees teach that it is generally preferred to have an excess of iodine over the amount of iodine which is present as a ligand with the rhodium component of the catalyst. Generally speaking, the teaching of the patentees appears to be that iodide which is added as, for example, an iodide salt, functions simply as a precursor component of the catalyst system. Where the patentees add hydrogen iodide, they regard it as a precursor of the promoter methyl iodide. There is no clear teaching that simple iodide ions as such are of any significance nor that it is desirable to have them present in substantial excess to increase the rate of the reaction. As a matter of fact, Eby and Singleton [Applied Industrial Catalysis, Vol. 1, 275-296(1983)] from Monsanto state that iodide salts of alkali metals are inactive as co-catalyst in the rhodium-catalyzed carbonylation of methanol.

Carbonylation of esters, such as methyl acetate, or ethers, such as dimethyl ether, to form a carboxylic acid anhydride such as acetic anhydride is disclosed in U.S. Pat. No. 4,115,444 issued to Rizkalla and in European Patent Application No. 0,008,396 by Erpenbach et al. and assigned to Hoechst. In both cases the catalyst system comprises rhodium, an iodide, and a trivalent nitrogen or phosphorus compound. Acetic acid can be a component of the reaction solvent system, but it is not the reaction product. Minor amounts of water are indicated to be acceptable to the extent that water is found in the commercially-available forms of the reactants. However, essentially dry conditions are to be maintained in these reaction systems. U.S. Pat. No. 4,374,070 issued to Larkins et al. teaches the preparation of acetic anhydride in a reaction medium which is, of course, anhydrous by carbonylating methyl acetate in the presence of rhodium, lithium, and an iodide compound. The lithium can be added as lithium iodide. Aside from the fact that the reaction is a different one from that with which the present invention is concerned, there is no teaching that it is important per se that the lithium be present in any particular form such as the iodide. There is no teaching that iodide ions as such are in significant amounts.

U.S. Pat. No. 5,001,259, U.S. Pat. No. 5,026,908 and U.S. Pat. No. 5,144,068 disclose a rhodium-catalyzed low water method for the production of acetic acid. Methanol is reacted with carbon monoxide in a liquid reaction medium containing a rhodium catalyst stabilized with an iodide salt, especially lithium iodide, along with alkyl iodide such as methyl iodide and alkyl acetate such as methyl acetate in specified proportions. This reaction system not only provides an acid product of unusually low water content (lower than 14 wt %) at unexpectedly favorable reaction rates but also, whether the water content is low or, as in the case of prior-art acetic acid technology, relatively high, is characterized by unexpectedly high catalyst stability, i.e., it is resistant to catalyst precipitation out of the reaction medium. Employing a low water concentration simplifies downstream processing of the desired carboxylic acid to its glacial form.

Various means for removing iodide impurities from acetic acid are well know in the art. It was discovered by Hilton that macroreticular strong acid cation exchange resins with at least one percent of their active sites converted to the silver or mercury form exhibited remarkable removal efficiency for iodide contaminants in acetic acid or other organic media. The amount of silver or mercury associated with the resin may be from as low as about one percent of the active sites to as high as 100 percent. Preferably about 25 percent to about 75 percent of the active sites were converted to the silver or mercury form and most preferably about 50 percent. The subject process is disclosed in U.S. Pat. No. 4,615,806 issued to Hilton for removing various iodides from acetic acid. In particular there is shown in the examples removal of methyl iodide, hydrogen iodide, I₂ and hexyl iodide.

Various embodiments of the basic invention disclosed in U.S. Pat. No. 4,615,806 have subsequently appeared in the literature. There is shown in U.S. Pat. No. 5,139,981 issued to Kurland a method for removing iodides from liquid carboxylic acid contaminated with a halide impurity by contacting the liquid halide contaminant acid with a silver (I) exchanged macroreticular resin. The halide reacts with the resin bound silver and is removed from the carboxylic acid stream. The invention in the '981 patent more particularly relates to an improved method for producing the silver exchanged macroreticular resins suitable for use in iodide removal from acetic acid.

U.S. Pat. No. 5,227,524 issued to Jones discloses a process for removing iodides using a particular silver-exchanged macroreticular strong acid ion exchange resin. The resin has from about 4 to about 12 percent cross-linking, a surface area in the proton exchanged form of less than 10 m²/g after drying from the water wet state and a surface area of greater than 10 m²/g after drying from a wet state in which the water has been replaced by methanol. The resin has at least one percent of its active sites converted to the silver form and preferably from about 30 to about 70 percent of its active sites converted to the silver form.

U.S. Pat. No. 5,801,279 issued to Miura et al. discloses a method of operating a silver exchanged macroreticular strong acid ion exchange resin bed for removing iodides from a Monsanto type acetic acid stream. The operating method involves operating the bed silver-exchanged resin while elevating the temperatures in stages and contacting the acetic acid and/or acetic anhydride containing the iodide compounds with the resin. Exemplified in the patent is the removal of hexyl iodide from acetic acid at temperatures of from about 25° C. to about 45° C.

So also, other ion exchange resins have been used to remove iodide impurities from acetic acid and/or acetic anhydride. There is disclosed in U.S. Pat. No. 5,220,058 issued to Fish et al. the use of ion exchange resins having metal exchanged thiol functional groups for removing iodide impurities from acetic acid and/or acetic anhydride. Typically, the thiol functionality of the ion exchange resin has been exchanged with silver, palladium, or mercury.

There is further disclosed in European Publication No. 0 685 445 A1 a process for removing iodide compounds from acetic acid. The process involves contacting an iodide containing acetic acid stream with a polyvinylpyridine at elevated temperatures to remove the iodides. Typically, the acetic acid was fed to the resin bed according to the '445 publication at a temperature of about 100° C.

With ever increasing cost pressures and higher energy prices, there has been ever increasing motivation to simplify chemical manufacturing operations and particularly to reduce the number of manufacturing steps. In this regard, it is noted that in U.S. Pat. No. 5,416,237 issued to Aubigne et al. there is disclosed a single zone distillation process for making acetic acid. Such process modifications, while desirable in terms of energy costs, tend to place increasing demands on the purification train. In particular, fewer recycles tend to introduce (or fail to remove) a higher level of iodides into the product stream and particularly more iodides of a higher molecular weight. For example, octyl iodide, decyl iodide and dodecyl iodides may all be present in the product stream as well as hexadecyl iodide; all of which are difficult to remove by conventional techniques.

The prior art resin beds operated as described above do not efficiently and quantitatively remove impurities from acetic acid or acetic acid streams as required by certain end consumers, particularly the manufacture of vinyl acetate monomer (VAM). Accordingly, there is still a need to remove the impurities to a desired amount in an acetic acid product stream.

### B. Formation of Impurities in Methanol Carbonylation

It has been found that during the production of acetic acid by the carbonylation of methanol or methyl acetate in the presence of a finite amount of water, carbonyl impurities such as acetaldehyde, acetone, methyl ethyl ketone, butyraldehyde, crotonaldehyde, 2-ethyl crotonaldehyde, and 2-ethyl butyraldehyde and the like, are present and may further react to form aldol condensation products and/or react with iodide catalyst promoters to form multi-carbon alkyl iodides, i.e., ethyl iodide, butyl iodide, hexyl iodide and the like. While the presence of hydrogen in the carbonylation reaction does in fact increase the carbonylation rate, the rate of formation of undesirable by-products, such as crotonaldehyde, 2-ethyl crotonaldehyde, butyl acetate, and hexyl iodide, also increases.

One prominent theory for the formation of the crotonaldehyde and 2-ethyl crotonaldehyde impurities in the methanol carbonylation process is that they result from aldol and cross-aldol condensation reactions that involve acetaldehyde. The possible reaction formula is as follows:

2 CH₃CHO(acetaldehyde)→CH₃CHCHCHO(crotonaldehyde)+H₂O(water)

The crotonaldehyde may be reduced to butyraldehyde in the presence of hydrogen and further reacts with acetaldehyde to produce 2-ethyl crotonaldehyde.

CH₃CH₂CH₂CHO(butyraldehyde)+CH₃CHO(acetaldehyde)→CH₃CHC(C₂H₅)CHO(2-ethyl crotonaldehyde)+H₂O(water)

The abovementioned butyraldehyde will react with hydrogen to generate butanol. Subsequently, the butanol will react with the acetic acid product to generate butyl acetate.

C₄H₉OH(butanols)+CH₃COOH(acetic acid)→CH₃COOC₄H₉(butyl acetate)+H₂O(water)

Furthermore, the use of iodide compounds in the production of acetic acid will remain in the acetic acid product.

### C Disadvantages of Impurities

Glacial acetic acid is a raw material for several key petrochemical intermediates and products including VAM, acetate esters, cellulose acetate, acetic anhydride, monochloroacetic acid (MCA), etc., as well as a key solvent in the production of purified terephthalic acid (PTA). Consumers of glacial acetic acid generally prefer a high purity product with as few impurities as possible and the lowest concentration on any contained impurities.

The iodide contamination can be of great concern to the consumers of the acetic acid as it may cause processing difficulties when the acetic acid is subjected to subsequent chemical conversion. A higher iodide environment could lead to increased corrosion problems and higher residual iodide in the final product. High iodide concentration in acetic acid could lead to catalyst poisoning problems in some downstream applications such as vinyl acetate monomer (VAM) manufacture.

### III. SUMMARY OF THE INVENTION

There is provided in a first aspect of the present invention, a method of controlling impurities in the product glacial acetic acid of a rhodium-catalyzed methanol carbonylation process for the manufacture of acetic acid, comprising:
a) reacting methanol with carbon monoxide in a liquid reaction medium in the presence of a Group VIII metal catalyst; and
b) maintaining in said reaction medium a water concentration of 0.5 to 14 wt %, an iodide salt providing an ionic iodide in the range of 2 to 20 wt %, 1 to 20 wt % methyl iodide and 0.5 to 30 wt % methyl acetate thereby an acetic acid is produced;
whereby the resulting product glacial acetic acid comprises total aldehyde in an amount greater than 2 ppm.

In another aspect of the present invention, a method of controlling impurities in a non-aqueous organic medium comprising further contacting the organic medium containing impurities with an ion exchange resin. The ion exchange resin is characterized in that it is a macroreticular strong-acid cation exchange resin which is stable in the organic medium and has at least one percent of its active sites converted to the silver or mercury form. The term “non-aqueous” simply means that water is not present to any significant extent. Generally, not present by more than 1%, and typically not present by more than 0.5% even in the case of organic media which are highly miscible with water. In one embodiment of the present invention, the non-aqueous organic medium is acetic acid produced by rhodium-catalyzed methanol carbonylation.

More specifically, there is provided in accordance with the present invention a method of removing impurities from a non-aqueous organic medium comprising further contacting the organic medium with a silver or mercury exchanged cation exchange substrate at a temperature greater than about 50° C. When a silver or mercury exchanged substrate is used, it is typically a macroreticular, strong acid cation resin. Temperatures may be from about 60 to about 100° C. A minimum temperature of 60° C. is sometimes employed while a minimum temperature of about 70° C. may likewise be preferred in some embodiments. In general, when a silver or mercury exchanged strong acid cation resin is employed typically from about 25% to about 75% of the active sites are converted to the silver or mercury form. Most typically about 50% of the active sites are so converted.

It has been discovered that impurities levels in glacial acetic acid product produced by rhodium-catalyzed methanol carbonylation can be controlled to certain levels by using the above-mentioned methods. Accordingly, there is provided in accordance with further aspect of the present invention, the product glacial acetic acid of a rhodium-catalyzed methanol carbonylation process is characterized by the presence of total aldehyde in an amount greater than 2 ppm and total iodide in an amount less than 150 ppb. In still other embodiment, the product glacial acetic acid of catalyzed methanol carbonylation process is characterized by the presence of total aldehyde in an amount greater than 2 ppm, total iodide in an amount less than 150 ppb and hexyl iodide in an amount less than 20 ppb. According to the invention, the amount of total aldehyde is preferably in an amount of from 5 ppm to 50 ppm. The amount of total iodide is preferably in an amount of from 1 ppb to 100 ppb. The amount of hexyl iodide is preferably in an amount of from 1 ppb to 10 ppb.

As used herein, glacial acetic acid is concentrated, higher than 99.5% pure acetic acid. Glacial acetic acid is called “glacial” because its freezing point (16.7° C.) is only slightly below room temperature. In the (generally unheated) laboratories in which the pure material was first prepared, the acid was often found to have frozen into ice-like crystals. The term “glacial acetic acid” is now taken to refer to pure acetic acid (ethanoic acid) in any physical state. Further, total aldehyde consists of those saturated and unsaturated aldehydes in the final acetic acid product that are formed in the methanol carbonylation process via aldol reactions derived from acetaldehyde with is also produced as an impurity in the process. The major aldehyde impurity components of the total aldehyde are crotonaldehyde and ethyl crotonaldehyde.

### IV. BRIEF DESCRIPTION OF THE DRAWINGS

FIG. 1 is a process flow diagram illustrating a typical rhodium-catalyzed methanol carbonylation process. FIG. 2 is a process flow diagram illustrating a typical rhodium-catalyzed methanol carbonylation process additionally equipped with a heavy ends column.

FIG. 3 is a process flow diagram illustrating a typical rhodium-catalyzed methanol carbonylation process additionally equipped with a heavy ends column and a guard bed.

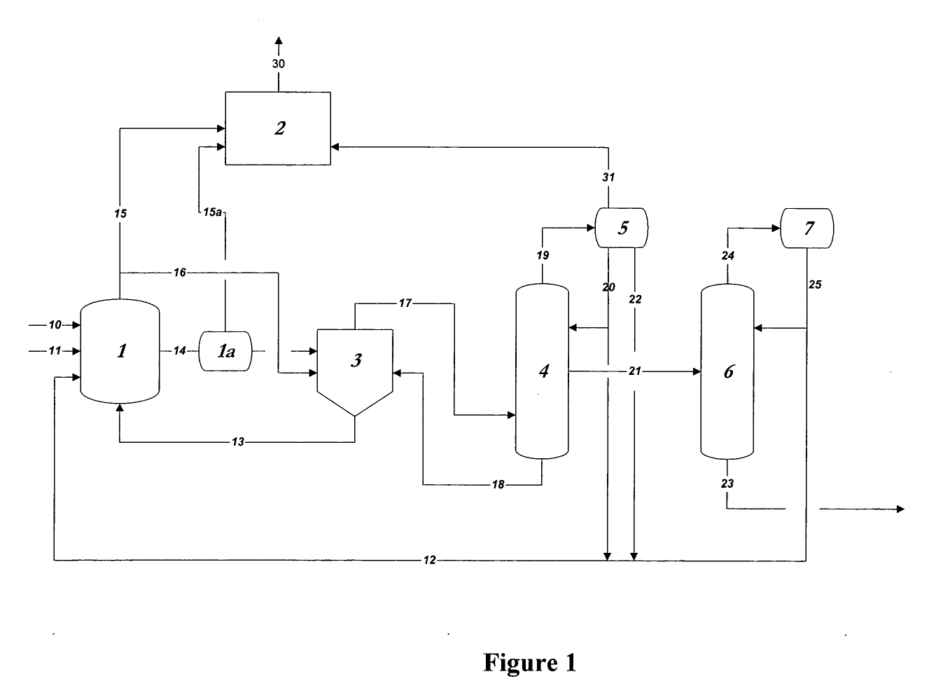

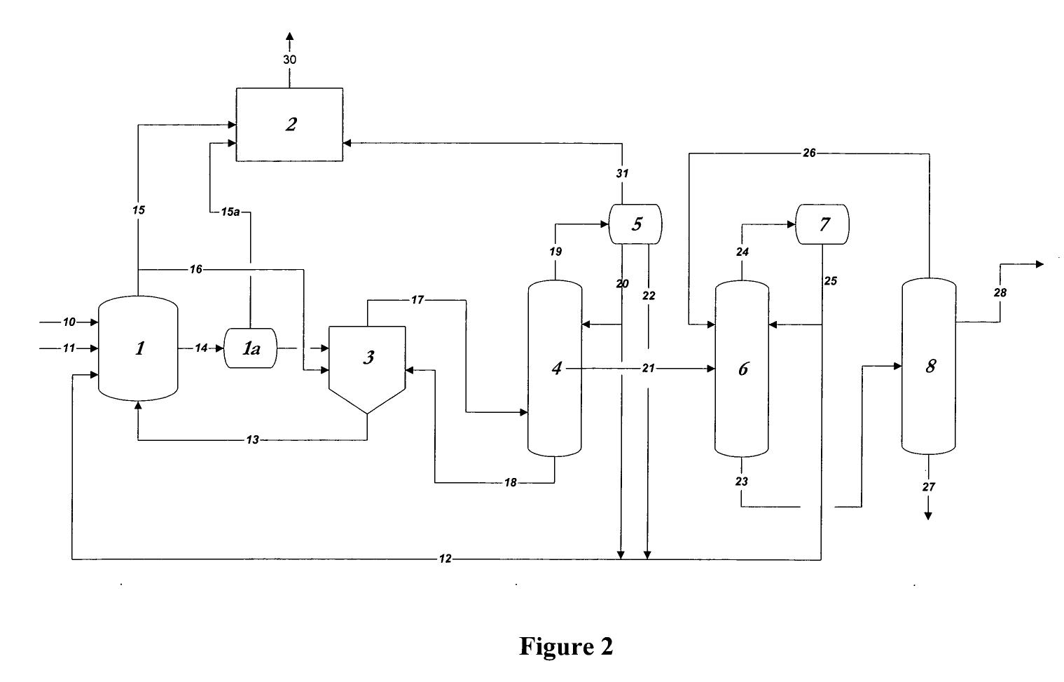

It is understood that FIGS. 1, 2 and 3 are merely typical examples of common flow patterns for a methanol carbonylation process. It is also understood that FIGS. 1, 2 and 3 are non-limiting to this invention and that there can be many alternative variations to these “typical” flow diagrams within the scope of this invention.

### V. DETAILED DESCRIPTION OF THE INVENTION

### A. General Rhodium-Catalyzed Methanol Carbonylation Reaction to Make Acetic Acid

To produce acetic acid by methanol carbonylation, methanol is reacted with carbon monoxide in the presence of a catalyst. The general formula is as follows:

CH₃OH+CO [symbol] CH₃COOH

In the practice of the present invention, rhodium is used as the catalyst in methanol carbonylation process and renders the process highly selective. Methyl iodide is used as a promoter and an iodide salt is maintained in the reaction medium to enhance stability of the rhodium catalyst. Water is also maintained from a finite amount up to 14 wt % in the reaction medium. A reaction system which can be employed, within which the present improvement is used, will be further explained below, comprises:
(a) a liquid-phase or slurry type carbonylation reactor which optionally may include a so-called “converter” reactor,
(b) a “flasher” vessel, and
(c) a purification system consisting of distillation and vent scrubbing using two or more columns to separate volatile components comprising methyl iodide, methyl acetate, water 30 and other light ends and generate a purified glacial acetic acid product.

### B. General Process Flow

### 1. Reactor

Referring to FIG. 1, the reactor, 1, is typically a stirred autoclave, bubble column reactor vessel or gas-liquid flow eduction vessel within which the reacting liquid or slurry contents are maintained automatically at a constant level. Carbon monoxide is fed via line 11 to the reactor. The carbon monoxide is thoroughly dispersed through the reacting liquid by such means as physical agitation, gas-liquid sparger diffusion, gas-liquid flow eduction or other known gas-liquid contacting techniques.

Into this reactor there are also continuously introduced fresh and recycled carbonylatable reactants (such as methanol, methyl acetate, dimethyl ether and/or mixtures thereof) via a methanol feed 10; a recycle stream 12 including water, methyl iodide and methyl acetate from the overhead of the light ends column 4 and the drying column 6; the catalyst recycle 13 from the base of the flasher 3, and optionally a fresh water makeup (if needed) to maintain at least a finite concentration of water in the reaction medium. For example, a continuous fresh water feed is needed to maintain a finite water concentration in the reaction medium when the feedstock is methyl acetate and/or dimethyl ether. When the feedstock is methanol, a continuous fresh water feed may or may not be needed depending upon the rate of water consumption via the known water gas shift reaction. Alternate distillation systems can be employed so long as they provide means for recovering a crude acetic acid and directly or indirectly recycling the reactor catalyst solution components such as methyl iodide, water, methyl acetate and rhodium. Carbon monoxide is also continuously introduced into the reactor.

A high pressure vent gas 15 is typically vented from the head of the reactor to prevent buildup of gaseous by-products such as methane, carbon dioxide and hydrogen and to maintain a set carbon monoxide partial pressure at a given total reactor pressure. A portion of the high pressure vent gas which contains carbon monoxide can also be optionally used as a purge, via line 16, to the flasher base liquid to enhance rhodium stability.

An optional converter la can be employed which is located between the reactor and flasher. If an optional converter is employed, the effluent from the reactor 1 is transferred to the converter through the reaction medium transfer line 14, and its effluent is transferred to the flasher 3 via line 16. Without the optional converter, the reactor 1 effluent would flow directly to the flasher 3. The high pressure vent gases from the reactor, line 15, and converter, line 15a, are typically scrubbed in the gas scrubbing system, 2, with a compatible solvent to recover components such as methyl iodide and methyl acetate. These vent gasses can be combined or scrubbed separately and are typically scrubbed with either acetic acid, methanol or mixtures of acetic acid and methanol to prevent loss of low boiling components such as methyl iodide from the process. If methanol is used as the vent scrub liquid solvent, the enriched methanol from the gas scrubbing system 2 is typically returned to the process by combining with the fresh methanol feeding the carbonylation reactor, although it can also be returned into any of the streams that recycle back to the reactor such as the flasher residue or light ends or drying column overhead streams. If acetic acid is used as the vent scrub liquid solvent, the enriched acetic acid from the scrubbing system is typically stripped of absorbed light ends and the resulting lean acetic acid is recycled back to the absorbing step. The light end components stripped from the enriched acetic acid scrubbing solvent can be returned to the main process directly or indirectly in several different locations including the reactor, flasher, or purification columns.

The non-condensable gaseous components from the reactor vent line 15 and purification system vent line 31 that are not recovered typically by scrubbing using acetic acid or methanol to capture and recover methyl iodide and other light boiling components from the vent streams, are purged from the plant via line 30. The enriched acetic acid or methanol scrub liquid containing the light components recovered from streams 15 and 31 are returned to the process thereby preventing loss of the valuable light boiling components comprising methyl iodide and methyl acetate.

Optionally, the vent gases may be vented through the flasher base liquid or lower part of the light ends column to enhance rhodium stability and/or they may be combined with other gaseous process vents (such as the purification column overhead receiver vents) prior to scrubbing. These variations are well known to those skilled in the art.

### 2. Flasher

Referring to FIG. 1, liquid product is drawn off from the reactor 1 via line 14 (or optional converter via line 16) at a rate sufficient to maintain a constant level therein and is introduced to the flasher, 3, at a point intermediate between the top and bottom thereof.

In the flasher the catalyst solution is withdrawn as a base stream 13 (predominantly acetic acid containing the rhodium and the iodide salt along with lesser quantities of methyl acetate, methyl iodide, and water), while the overhead of the flasher comprises largely crude acetic acid along with methyl iodide, methyl acetate, and water, as well as any dissolved gasses that were contained in the reactor effluent transferred to the flasher. This stream is fed to the light ends column, 4, via line 17.

### 3. Purification—Light Ends Column, Drying Column, Heavy Ends Column and Guard Bed

Referring to FIGS. 1, 2 and 3, the crude acetic acid is typically drawn as a side stream near the base of the light ends column 4 via line 21 for further water removal in a drying column 6. The overhead distillate of the light ends column typically comprises water; methyl iodide, methyl acetate and some acetic acid. It is common that the light ends overhead vapor stream 19 is condensed and then separated through a light ends column decanter 5 into two phases consisting of a predominately aqueous phase 20 and a predominately organic phase 22. Both phases are directly or indirectly recycled back into the reaction medium. A residue stream can be taken from the light ends column which may contain some traces of rhodium catalyst entrained from the flasher vessel. The residue stream from the light ends column is typically returned to the flasher vessel or reaction medium via line 18, thereby returning the entrained rhodium and other entrained catalyst components.

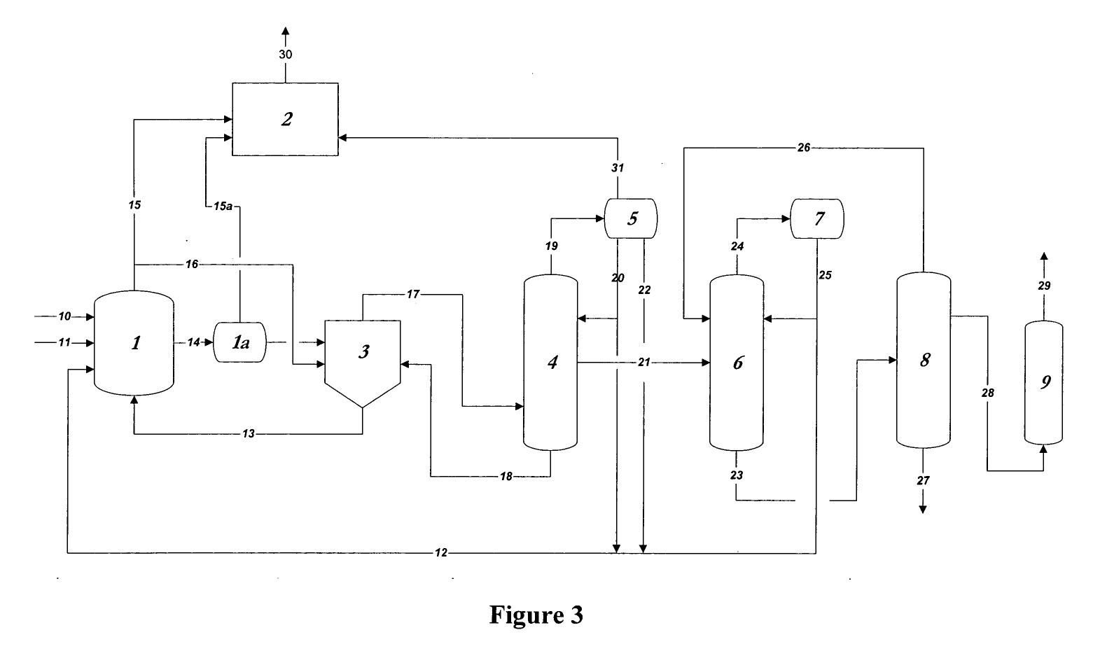

The crude acetic acid from the light ends column is further distilled in the drying column 6 to primarily remove the remaining water, methyl iodide and methyl acetate as an overhead distillate. The overhead vapor from the drying column is sent to a drying column reflux drum 7 via line 24. The net condensed overhead of the drying column is also recycled directly or indirectly back to the reaction medium typically via line 25. The residue 23 of the drying column is, as the column name implies, dry acetic acid. As shown in FIGS. 2 and 3 it can be further treated if necessary to remove impurities such as propionic acid, crotonaldehyde, 2-ethyl-crotonaldehyde and butyl acetate in a heavy ends column 8 or to remove impurities such as iodides in a guard bed 9, or it can be treated directly by a “polishing” system to remove specific trace impurities such as iodides. The overhead product from the heavy ends column is transferred back to the drying column via line 26. The heavy byproduct 27 of the heavy ends column is purged. The final glacial acetic acid product can be the “polished” drying column residue or it can be a distillate or sidestream from the heavy ends column, shown as line 28 in FIG. 2 or a product 29 from the guard bed, as shown in FIG. 3. Simple variations on the final purification are obvious to those skilled in the art and are outside the scope of the present invention.

### C. Reaction Condition

### 1. Temperatures & Pressures

The temperature of the reactor is controlled automatically, and the carbon monoxide is introduced at a rate sufficient to maintain a constant total reactor pressure. The carbon monoxide partial pressure in the reactor is typically about 2 to 30 atmospheres absolute, preferably about 4 to 15 atmospheres absolute. Because of the partial pressure of by-products and the vapor pressure of the contained liquids, the total reactor pressure is from about 15 to 45 atmospheres absolute, with the reaction temperature being approximately 150° C. to 250° C. Preferably, the reactor temperature is about 175° C. to 220° C.

### 2. Reaction Rates

The rate of the carbonylation reaction according to the present state of the art has been highly dependent on water concentration in the reaction medium, as taught by U.S. Pat. No. 3,769,329; EP0055618; and Hjortkjaer and Jensen (1977). That is, as the water concentration is reduced below about 14-15 wt % water, the rate of reaction declines. The catalyst also becomes more susceptible to inactivation and precipitation when it is present in process streams of low carbon monoxide partial pressures. It has now been discovered, however, that increased acetic acid-production capacity can be achieved at water concentrations below about 14 wt % (at water contents above about 14 wt %, the reaction rate is not particularly dependent on water concentration) by utilizing a synergism which exists between methyl acetate and the iodide salt as exemplified by lithium iodide especially at low water concentrations.

### D. Reaction Medium

### 1. Group VIII Metal Catalyst

The carbonylation between carbon monoxide and methanol is conducted in the presence of a Group VIII metal catalyst. Preferably, the Group VIII metal catalyst is rhodium and iridium. For example, the rhodium complex [RhI₂(CO)₂]— as a catalyst to prepare acetic acid. The concentration of rhodium catalyst used in the invention is about 200 ppm to about 2000 ppm.

### 2. Ranges of Components

a) Methyl Iodide

Methyl iodide is a promoter of rhodium catalyst and its concentration is relevant to the reaction rate. The concentration of reactor methyl iodide used in the experiments mentioned in the invention was maintained between about 5 wt % and 20 wt % during the course of the experiments. If the concentration of methyl iodide is higher than 20 wt %, rhodium catalyst will be precipitated at an accelerated rate, which thus causes a loss of rhodium catalyst and increases the load of the downstream purification procedures as well as decreases the productivity. However, a concentration of methyl iodide less than 5 wt % reduces much of the effectiveness to promote the rhodium catalyst and thus decreases the reaction rate. Therefore, the concentration of methyl iodide in the reactor of the invention should be maintained within the range between 5 wt % and 20 wt %.

b) Methyl Acetate

Methyl acetate will be formed in situ by the esterification of methanol and acetic acid. The concentration of methyl acetate is relevant to the reaction rate of methanol carbonylation and should be maintained in a proper range to provide an optimum reaction rate. High methyl acetate concentration causes precipitation and loss of rhodium catalyst. Further, if the concentration of methyl acetate is maintained below 0.5 wt %, the reaction rate will be too low to be economical. Therefore, the concentration of methyl acetate in the reactor is maintained in the range between 0.5 wt % and 30 wt %.

c) Water

According to the invention, the reactor water concentration ranges from 0.5 wt % to 14 wt %. Preferably, the reactor water concentration ranges from 0.5 wt % to 8 wt % and more preferably 0.5 wt % to 4 wt %.

### 3. Iodides

The iodide(s) used in the invention for conducting the carbonylation reaction to prepare acetic acid are iodide salts and methyl iodide. Maintaining iodide salts in the reaction medium is the most effective way to stabilize the rhodium catalyst in the methanol carbonylation reaction. The invention utilizes iodide salts to maintain iodide ions in an amount of 2 wt % to 20 wt % in the carbonylation reaction for preparing acetic acid. The iodide ions can be formed directly by adding soluble iodide salts or they can be formed in-situ by the addition or accumulation of various non-iodide salts such as metal acetates, hydroxides, carbonates, bicarbonates, methoxides and/or amines, phosphines, stilbines, arsenes, sulfides, sulfoxides or other compounds that are capable of generating iodide ions in the reaction medium through reaction with methyl iodide or HI. Non-limiting examples would include compounds such as lithium acetate, lithium hydroxide, lithium carbonate, potassium hydroxide, potassium iodide, potassium acetate, sodium hydroxide, sodium carbonate, sodium bicarbonate, sodium methoxide, calcium carbonate, magnesium carbonate, pyridine, imidazole, triphenyl phosphine, triphenyl phosphine oxide, dimethyl sulfide, dimethyl sulfoxide, polyvinyl pyridine, polyvinyl pyridine N-oxide, methylpyridinnium iodide and polyvinyl pyrrolidone.

### E. Control of Impurities in Product Glacial Acetic Acid of Rhodium-Catalyzed Methanol Carbonylation

As discussed above, the present invention provides a method of controlling impurities in the product glacial acetic acid of a rhodium-catalyzed methanol carbonylation process for the manufacture of acetic acid. According to the invention, the control method comprises reacting methanol with carbon monoxide in the presence of a Group VIII metal catalyst in a reaction vessel; and maintaining in said reaction vessel a water concentration of 0.5 to 14 wt %, an iodide salt providing an ionic iodide in the range of 2 to 20 wt %, 1 to 20 wt % methyl iodide and 0.5 to 30 wt % methyl acetate thereby an acetic acid is produced; whereby the resulting glacial acetic acid product comprises total aldehyde in an amount greater than 2 ppm.

The present invention further provides a method of controlling impurities in the product glacial acetic acid by contacting the resulting acetic acid with a silver or mercury exchanged cation exchange substrate at a temperature greater than about 50° C. When a silver or mercury exchanged substrate is used, it is typically a macroreticular, strong acid cation exchange resin. Temperatures may be from about 60 to about 100° C. A minimum temperature of 60° C. is sometimes employed while a minimum temperature of about 70° C. may likewise be preferred in some embodiments. In general, when a silver or mercury exchanged strong acid cation exchange resin is employed typically from about 25% to about 75% of the active sites are converted to the silver or mercury form. Most typically about 50% of the active sites are so converted. The resulting product glacial acetic acid may further contain total iodide in an amount of less than 150 ppb, or hexyl iodide in an amount less than 20 ppb.

Ion exchange resins or other suitable substrates are typically prepared for use in connection with the present invention by exchanging anywhere from about 1 to about 99 percent of the active sites of the resin to the silver or mercury salt, as is taught for example in U.S. Pat. Nos. 4,615,806; 5,139,981; 5,227,524 the disclosures of which are hereby incorporated by reference.

Suitable stable ion exchange resins utilized in connection with the present invention typically are of the “RSO₃H type” classified as “strong acid”, that is sulfonic acid, cation exchange resins of the macroreticular (macroporous) type. Particularly suitable ion exchange substrates include Amberlyst® 15 resin (Rohm and Haas), being particularly suitable for use at elevated temperatures. Other stable ion exchange substrates such as zeolites may be employed, provided that the material is stable in the organic medium at the conditions of interest, that is, will not chemically decompose or release silver or mercury into the organic medium in unacceptable amounts. Zeolite cation exchange substrates are disclosed for example, in U.S. Pat. No. 5,962,735 issued to Kulprathipanja et al., the disclosure of which is incorporated herein by reference.

The process of the present invention may be carried out in any suitable configuration. A particularly preferred configuration is to utilize a bed of particulate material (termed herein a “guard bed”) inasmuch as this configuration is particularly convenient. A typical flow rate, such as is used when acetic acid is to be purified, is from about 0.5 to about 20 bed volumes per hour (BV/hr). A bed volume of organic medium is simply a volume of the medium equal to the volume occupied by the resin bed. A flow rate of 1 BV/hr then means that a quantity of organic liquid equal to the volume occupied by the resin bed passes through the resin bed in a one hour time period. Preferred flow rates are usually from about 6 to about 10 BV/hr whereas a preferred flow rate is frequently about 6 BV/hr.

The present invention is further described in connection with FIGS. 1, 2 and 3. The apparatus includes a reactor, a flasher, a light ends column, a drying column, a heavy ends column and a guard bed. Crude acetic acid product is manufactured by rhodium-catalyzed methanol carbonylation as previously described. The acetic acid product is withdrawn from the drying column and fed to the heavy ends column and then to the guard bed used for controlling impurities in the product glacial acetic acid of rhodium-catalyzed methanol carbonylation. The resin bed is a bed of silver or mercury exchanged cation exchange media and is typically operated at an average product temperature of greater than about 50° C.

The present invention is better understood by reference to the following examples. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### VI. EXAMPLES

### A. Example 1

The following experimental runs were carried out in a continuously operating system comprising the equipment and components previously described hereinabove. The liquid reaction medium in the reactor was maintained between 7 and 13 wt % methyl iodide, 1 to 3.2 wt % methyl acetate, 0.4 to 11.5 wt % iodide ion, 1.7 to 14.6 wt % water, and 500 to 1300 ppm of rhodium. The balance of the reaction medium was essentially acetic acid.

During experiments, the reactor temperature was maintained between about 189 to 199° C. The pressure was maintained at about 26 to 28 atmospheres absolute. Carbon monoxide was continuously introduced through a sparger situated below the mechanical agitator blades, and a continuous vent of gas was drawn off from the top of the vapor space contained in the upper part of the reactor. The reactor vent and other non-condensable gases collected from the purification train were scrubbed with acetic acid to prevent losses of methyl iodide and other low boiling components contained in the vent streams. The light end components from the acetic acid scrubbing system were continuously returned to the process and the low boiling components in the vent streams were thus retained in the process. The carbon monoxide partial pressure in the reactor headspace was maintained at about 4 to 9 atmospheres absolute.

By means of a level control sensing the liquid level within the reactor, liquid reaction product was continuously drawn off and fed into a flasher vessel operating at a head pressure of about 3 atmospheres absolute. The vaporized portion of the introduced catalyst liquid exiting the overhead of the flasher was distilled in the light ends column.

The light ends column was used to separate and recycle primarily methyl iodide, methyl acetate and a portion of the water from the crude acetic acid. A sidestream from the light ends column was drawn off as the crude acetic acid to feed a drying column for further purification.

A drying column was then used to remove the remaining water, methyl iodide and methyl acetate from the crude acetic acid. The distillate of the drying column was combined with the distillate from the light ends column and recycled back to the reaction section. Results are shown in Table 1 below.

TABLE 1
Impurities in Product Glacial Acetic Acid of Rhodium-Catalyzed
Methanol Carbonylation

Contents in
product from
the drying column

| Impurities | Run 1 | Run 2 |
|---|---|---|
| Total Aldehyde*, ppm | 23 | 32 |
| Total Iodide**, ppb | 540 | 238 |
| Hexyl-Iodide, ppb | N.D. | 30 |

*The total aldehyde consists of those saturated and unsaturated aldehydes in the final acetic acid process that are formed in the methanol carbonylation process via aldol reactions derived from acetaldehyde with is also produced as an impurity in the process. The major aldehyde impurity components of the total aldehyde are crotonaldehyde and ethyl crotonaldehyde.
**The total iodide was identified to include: methyl iodide, ethyl iodide, 2-iodo-2-methyl propane, propyl iodide, 2-butyl iodide, butyl iodide, iodine, pentyl iodide, hexyl iodide, octyl iodide, decyl iodide, dodecyl iodide and hexadecyl iodide.

While the foregoing examples demonstrate the reduction of total aldehyde and the like, it will be appreciated by one of skill in the art that other impurities and byproducts in rhodium catalyzed carbonylation systems include butanol, butyl acetate, butyl iodide, ethanol, ethyl acetate, ethyl iodide, hexyl iodide and high boiling impurities. The present invention appears to minimize production of these impurities as well.

### B. Example 2

Following the procedure outlined above in Example 1, the residue of the drying column was further fed to a heavy ends column as shown in FIG. 2 where the heavy ends (primarily propionic acid) and the impurities such as the total aldehyde were removed in the residue and the distilled product glacial acetic acid was measured for impurities contents. Results are shown in Table 2 below. As can be seen from Table 2, the impurities such as total aldehyde and total iodides were significantly reduced by using a heavy ends column.

TABLE 2
Impurities in Product Glacial Acetic Acid of Rhodium-Catalyzed
Methanol Carbonylation

Contents in
product from
the heavy
ends column

| Impurities | Run 1 | Run 2 |
|---|---|---|
| Total Aldehyde*, ppm | 19 | 23 |
| Total Iodide**, ppb | 130 | 76 |
| Hexyl-Iodide, ppb | N.D. | 18 |

*The total aldehyde consists of those saturated and unsaturated aldehydes in the final acetic acid process that are formed in the methanol carbonylation process via aldol reactions derived from acetaldehyde with is also produced as an impurity in the process. The major aldehyde impurity components of the total aldehyde are crotonaldehyde and ethyl crotonaldehyde.
**The total iodide was identified to include: methyl iodide, ethyl iodide, 2-iodo-2-methyl propane, propyl iodide, 2-butyl iodide, butyl iodide, iodine, pentyl iodide, hexyl iodide, octyl iodide, decyl iodide, dodecyl iodide and hexadecyl iodide.

### C. Example 3

Following the procedure outlined above in Example 2, the residue of the heavy ends column was further fed to a guard bed as shown in FIG. 3 where samples of the residue were treated with a silver exchanged bed of Amberlyst® 15 resin. The resin (100 ml wet) was loaded into a 22 mm OD glass column and acetic acid containing impurities was eluted at a flow rate of 13.3 ml/min. Impurities levels in the eluate were measured every two (2) hours. Total iodide is measured in the eluate by any suitable technique. One suitable technique is by way of neutron activation analysis (NAA) as is well known in the art. The iodide levels for particular species were also measured. A preferred method in this latter respect is gas chromatography utilizing an electron capture detector. Results appear in Table 3 below. As can be seen from Table 3, the impurities such as total iodide and hexyl-iodide were significantly reduced by using the guard bed.

TABLE 3
Impurities in Product Glacial Acetic Acid of Rhodium-Catalyzed
Methanol Carbonylation

Contents in
product from
the guard bed

| Impurities | Run 1 | Run 2 |
|---|---|---|
| Total Aldehyde*, ppm | 19 | 23 |
| Total Iodide**, ppb | <10 | <10 |
| Hexyl-Iodide, ppb | N.D. | <5 |

*The total aldehyde consists of those saturated and unsaturated aldehydes in the final acetic acid process that are formed in the methanol carbonylation process via aldol reactions derived from acetaldehyde with is also produced as an impurity in the process. The major aldehyde impurity components of the total aldehyde are crotonaldehyde and ethyl crotonaldehyde.
**The total iodide was identified to include: methyl iodide, ethyl iodide, 2-iodo-2-methyl propane, propyl iodide, 2-butyl iodide, butyl iodide, iodine, pentyl iodide, hexyl iodide, octyl iodide, decyl iodide, dodecyl iodide and hexadecyl iodide.

While the present invention has been described in detail and exemplified, various modifications will be readily apparent to those of skill in the art. For example, one may utilize an ion exchange resin suited for higher temperatures in connection with the present invention. Such modifications are within the spirit and scope of the present invention which is defined in the appended claims.

What is claimed is:

1. A product glacial acetic acid of a rhodium-catalyzed methanol carbonylation acetic acid manufacturing process characterized by the presence of total aldehyde in an amount greater than 2 ppm.

2. The product glacial acetic acid of claim 1 wherein said product glacial acetic acid is further characterized by the presence of total iodide in an amount of less than 150 ppb.

3. The product glacial acetic acid of claim 1 wherein said product glacial acetic acid is further characterized by the presence of total iodide in an amount of from 1 ppb to 100 ppb.

4. The product glacial acetic acid of claim 1 wherein said product glacial acetic acid is further characterized by the presence of hexyl-iodide in an amount less than 20 ppb.

5. The product glacial acetic acid of claim 1 wherein said product glacial acetic acid is further characterized by the presence of hexyl-iodide in an amount of from 1 ppb to 10 ppb.

6. A method of controlling impurities in a rhodium-catalyzed methanol carbonylation process for the manufacture of acetic acid, comprising:
a) reacting methanol with carbon monoxide in a liquid reaction medium in the presence of a Group VIII metal catalyst;
b) maintaining in the reaction medium a water concentration of 0.5 to 14 wt %, an iodide salt providing an ionic iodide in the range of 2 to 20 wt %, 1 to 20 wt % methyl iodide and 0.5 to 30 wt % methyl acetate thereby an acetic acid is produced; and
c) contacting the resulting acetic acid stream with a silver or mercury exchanged cation exchange resin at a temperature greater than 50° C.;
d) whereby the resulting product glacial acetic acid comprises total aldehyde in an amount greater than 2 ppm.

7. The method of claim 6 wherein the water concentration is in an amount of 0.5 to 10 weight percent.

8. The method of claim 6 wherein the water concentration is in an amount of 0.5 to 8 weight percent.

9. The method of claim 6 wherein the water concentration is in an amount of 0.5 to 4 weight percent.

10. The method of claim 6 wherein the resulting product glacial acetic acid further contains total iodide in an amount of less than 150 ppb.

11. The method of claim 6 wherein the resulting product glacial acetic acid further contains total iodide in an amount of from 1 ppb to 100 ppb.

12. The method of claim 6 wherein the resulting product glacial acetic acid further contains hexyl-iodide in an amount less than 20 ppb.

13. The method of claim 6 wherein the resulting product glacial acetic acid further contains hexyl-iodide in an amount of from 1 ppb to 10 ppb.

14. The method of claim 6 wherein at least 1 percent of the active sites of said resin have been converted to the silver or mercury form.

15. The method of claim 6 wherein from 25 to 75% of the active sites of said resin have been converted to the silver or mercury form.

16. The method of claim 6 wherein said resin is a silver exchanged cation exchange resin.

17. The method of claim 6 wherein the Group VIII metal catalyst is a rhodium catalyst.

18. The acetic acid produced by the method of claim 6.

19. The acetic acid produced by the method of claim 7.

20. The acetic acid produced by the method of claim 8.

21. The acetic acid produced by the method of claim 9.

22. The acetic acid produced by the method of claim 10.

23. The acetic acid produced by the method of claim 11.

24. The acetic acid produced by the method of claim 12.

25. The acetic acid produced by the method of claim 13.

26. The acetic acid produced by the method of claim 14.

27. The acetic acid produced by the method of claim 15.

28. The acetic acid produced by the method of claim 16.

29. The acetic acid produced by the method of claim 17.
